(19) United States
(12) Patent Application Publication
(10) Pub. No.: US 2025/0027917 A1
(43) Pub. Date: Jan. 23, 2025

(54) SYSTEM FOR AUTOMATED PROCESSING OF MASS SPECTROMETRY SAMPLES AND DATA

(71) Applicants: Delaware State University, Dover, DE (US); The United States of America, as represented by the Secretary of Agriculture, Manhattan, KS (US)

(72) Inventors: Chase Stratton, Dover, DE (US); William Robert Morrison, III, Manhattan, KS (US); Ebony Murrell, Newark, DE (US); Yvonne Thompson, Newark, DE (US); Konilo Zio, Newark, DE (US)

(73) Assignees: Delaware State University, Dover, DE (US); The United States of America, as represented by the Secretary of Agriculture, Manhattan, KS (US)

(21) Appl. No.: 18/776,910

(22) Filed: Jul. 18, 2024

Related U.S. Application Data
(60) Provisional application No. 63/527,782, filed on Jul. 19, 2023.

(51) Int. Cl. G01N 30/86; G16C 20/60

(52) U.S. Cl. CPC G01N 30/8675; G01N 30/8651; G16C 20/60

(57) ABSTRACT

A system includes a processor, a network interface, an output coupled to the processor, and a memory coupled to the processor. The memory stores an idealized mass spectrogram library including a plurality of idealized mass spectrograms, each associated with an idealized compound, and a reference compound library including a plurality of reference compound identifiers, each associated with a reference structural datum. The system matches a sample mass spectrogram to one or more tentative idealized mass spectrograms; matches the idealized compound identifier associated with a tentative idealized mass spectrogram to a matching reference compound identifier of the plurality of reference compound identifiers; accepts a filtering structural datum; selects the matching reference compound identifiers matched to the idealized compounds associated with the one or more tentative idealized mass spectrograms with the reference structural datum satisfying the filtering structural datum; and outputs the selected matching reference compound identifiers.

### CROSS-REFERENCE TO RELATED APPLICATIONS

This application claims priority from U.S. Provisional Application Ser. No. 63/527,782, filed Jul. 19, 2023, titled SYSTEM FOR AUTOMATED PROCESSING OF MASS SPECTROMETRY SAMPLES AND DATA, and incorporated herein by reference.

### GOVERNMENT RIGHTS

This invention was made with government support under the following grant numbers: 2021-67034-35135 and 2018-67013-27402, both awarded by the U.S. Department of Agriculture, National Institute of Food and Agriculture (NIFA). The government has certain rights in the invention.

### FIELD OF THE INVENTION

This invention relates to systems and methods for identifying the chemical composition of samples, and more specifically, to automated processing of mass spectrometry samples and data.

### BACKGROUND OF THE INVENTION

Gas chromatography coupled with mass spectrometry (GC-MS), used to identify the chemical composition of samples, is a commonly used technology across many disciplines of research. While the accuracy and efficiency of instruments continues to improve, preparing the library-matched output, i.e., top hit(s) for each set of molecular fragments streamed across the machine's m/z detector, for analysis and interpretation remains antiquated.

The traditional methods involve manually selecting, integrating, and identifying peaks based on a reference library and comparison to commercial standards across every sample in an experiment. Software that identifies top library matches for every tentative compound in an entire batch of experimental samples exists; however, the output remains uninterpretable without additional process. In even simple experiments, the process of quantifying tentatively “identified” compounds across replicates can take weeks or months and can be a significant impediment to collecting and analyzing many, large, and/or complex GC-MS datasets. Furthermore, focusing the interpretation on specific chemicals or chemistries that are meaningful would require looking up each molecule for published information and/or important associations. This additional bottleneck in chemical experimentation can lead to backlogs in collections, delays in chemical data being analyzed and published, and may even create a significant deterrent to collecting GC-MS data in studies (e.g. non-targeted and/or suspect screening analysis) where these data could be highly informative.

Another concern with manually selecting component areas for the same tentative molecule across different samples is the inherent subjectivity and inconsistency at many decision points. Every additional keystroke or choice about threshold can provide an opportunity for unintended error.

Accordingly, there is a need in the field to address these barriers in the use of GC-MS data.

### SUMMARY OF THE INVENTION

The following presents a simplified summary of the invention in order to provide a basic understanding of some example aspects of the invention. This summary is not an extensive overview of the invention. Moreover, this summary is not intended to identify critical elements of the invention or to delineate the scope of the invention. The sole purpose of the summary is to present some concepts in a simplified form as a prelude to the more detailed description that is presented later.

In accordance with an aspect of the present invention, a system comprises a fluid chromatographer, which includes a mass spectrometer. The fluid chromatographer is configured to accept a sample and produce a sample chromatogram. The sample chromatogram describes at least a portion of the sample. The portion of the sample is comprised of one or more compounds to be identified. The sample chromatogram includes a plurality of sample mass spectrograms. The system further comprises a processor, an output coupled to the processor, and a memory coupled to the processor. The memory includes an idealized mass spectrogram library and a reference compound library. The idealized mass spectrogram library includes a plurality of idealized mass spectrograms, and each idealized mass spectrogram of the plurality of idealized mass spectrograms is associated with an idealized compound. The reference compound library includes a plurality of reference compound identifiers, and each reference compound identifier of the plurality of reference compound identifiers is associated with a reference structural datum. The system also includes programming in the memory, wherein execution of the programming by the processor configures the system to perform functions. The system matches a sample mass spectrogram of the plurality of sample mass spectrograms to one or more tentative idealized mass spectrograms of the plurality of idealized mass spectrograms. The system matches the idealized compound identifier associated with a tentative idealized mass spectrogram of the one or more tentative idealized mass spectrograms to a matching reference compound identifier of the plurality of reference compound identifiers. The system accepts a filtering structural datum. The system selects the matching reference compound identifiers matched to the idealized compounds associated with the one or more tentative idealized mass spectrograms with the reference structural datum satisfying the filtering structural datum. The system outputs, via the output, the selected matching reference compound identifiers corresponding to the one or more compounds to be identified.

According to another aspect of the invention, a system comprises a processor, a network interface, an output coupled to the processor, and a memory coupled to the processor. The memory includes an idealized mass spectrogram library and a reference compound library. The idealized mass spectrogram library includes a plurality of idealized mass spectrograms, and each idealized mass spectrogram of the plurality of idealized mass spectrograms is associated with an idealized compound. The reference compound library includes a plurality of reference compound identifiers, and each reference compound identifier of the plurality of reference compound identifiers is associated with a reference structural datum. The system also includes programming in the memory, wherein execution of the programming by the processor configures the system to perform functions. The system receives a sample chromatogram describing a sample, the sample chromatogram including a plurality of sample mass spectrograms. The system sends, via the network interface, a sample mass spectrogram of the plurality of sample mass spectrograms. The system receives, via the network interface, one or more tentative idealized mass spectrograms, the one or more tentative idealized mass spectrograms matching a sample mass spectrogram of the plurality of sample mass spectrograms. The system sends, via the network interface, a tentative idealized mass spectrogram of the one or more tentative idealized mass spectrograms. The system receives, via the network interface, an idealized compound, the idealized compound matching the tentative idealized mass spectrogram. The system accepts a filtering structural datum. The system sends, via the network interface, the idealized compound and the filtering structural datum. The system receives, via the network interface, a selected matching reference compound, the selected matching reference compound matched to the idealized compound and satisfying the filtering structural datum. The system outputs, via the output, the selected matching reference compounds.

Still another aspect of the invention includes a method for identifying one or more compounds. The method comprises matching a sample mass spectrogram of a plurality of sample mass spectrograms to one or more tentative idealized mass spectrograms of a plurality of idealized mass spectrograms. The method further comprises matching an idealized compound identifier associated with a tentative idealized mass spectrogram of the one or more tentative idealized mass spectrograms to a matching reference compound identifier of a plurality of reference compound identifiers. The method further comprises accepting a filtering structural datum. The method further comprises selecting the matching reference compound identifiers matched to the idealized compounds associated with the one or more tentative idealized mass spectrograms with the reference structural datum satisfying the filtering structural datum. The method further comprises outputting, via an output, the selected matching reference compound identifiers corresponding to the one or more compounds to be identified.

### BRIEF DESCRIPTION OF THE DRAWINGS

The foregoing and other aspects of the present disclosure will become apparent to those skilled in the art to which the present disclosure relates upon reading the following description with reference to the accompanying drawings, in which:

FIG. 1 illustrates correlations between volume of standards tests via GC/MS and the peak area estimates generated by uafR, for (A) Ethyl hexanoate, (B) Methyl salicylate, (C) Octanal, and (D) Undecane.

FIG. 2 is a schematic diagram illustrating an overall exemplary method for identifying the chemical composition of samples, according to an exemplary embodiment of the invention.

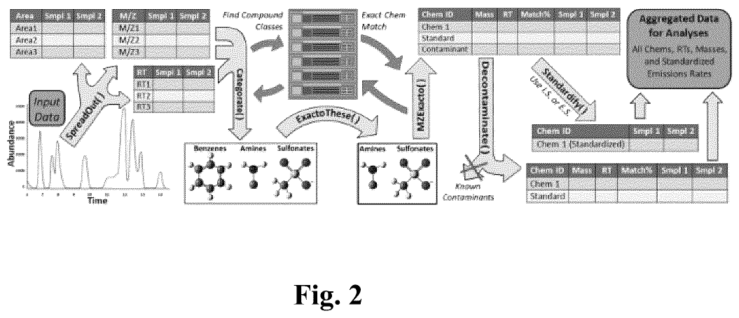

FIG. 3 is a flowchart illustrating an overall exemplary method for identifying the chemical composition of samples, according to an exemplary embodiment of the invention.

### DETAILED DESCRIPTION OF THE INVENTION

The invention will now be described by reference to exemplary embodiments and variations of those embodiments. Although the invention is illustrated and described herein with reference to specific embodiments, the illustrated examples are not intended to be limited to the details shown and described. Rather, various modifications may be made in the details within the scope and range of equivalents of the claims and without departing from the invention. For example, one or more aspects of the disclosed embodiments can be utilized in other embodiments and even other types of devices. Moreover, certain terminology is used herein for convenience only and is not to be taken as a limitation.

The following terminology is used throughout the foregoing description and in the appended claims:

| | |
|---|---|
| R | Programming language for statistical computing and |
| | graphics |
| NIST | National Institute of Standards and Technology |
| Exposomics | The study of environmental expires that an individual |
| | encounters throughout life |
| Metabolomics | The study of chemical processes associated with the |
| | immediate or end products of metabolism |
| Volatilomes | A volume containing all of the volatile metabolites as |
| | well as other volatile organic and inorganic compounds |
| | that originate from an organism or ecosystem |
| Chemotyping | The act of typing the chemically distinct entities in an |
| | organism with differences in the composition of the |
| | secondary metabolites |
| Component Area | Quantity of a specific chemical detected by a |
| | chromatographer or mass spectrometer |
| Match Factor | A dimensionless quantity describing the |
| | probability of a correct match between a sample |
| | compound and a reference compound |
| Tanimoto | A similarity measure for |
| | comparing chemical structures |

Described herein is a software package, uafR, based on an R programming language for statistical computing and data visualization or graphics (“R”), that can automate the demanding retrieval process for gas-chromatography coupled mass spectrometry (GC-MS) data and allow anyone interested in chemical comparisons to quickly perform advanced structural similarity matches. The streamlined cheminformatics workflows can allow anyone with basic experience in R to pull out component areas for tentative compound identifications using the best published understanding of molecules across samples in publicly accessible open chemistry databases, such as PubChem (e.g., pubchem.gov) maintained by the National Institutes of Health (NIH). Interpretations with uafR can be performed at a fraction of the time, cost, and effort typically taken using a standard chemical ecology data analysis pipeline.

The uafR package described herein can automate the sorting and collection of component areas across samples in an experiment while simultaneously storing critical information about every tentative molecule, and can propel every field of science forward by not only removing the bottlenecks and subjectivity in chemical analysis but also removing the need for hours of paid or untrained manual labor before even simple chemical interpretations can occur.

The uafR package takes the raw, aggregated chemical identifications generated from a user-selected peak detection software, such as Agilent's Unknowns Analysis software, for example, although any mass spectrometry software that produces the same information can be equally viable. The package described herein communicates with public chemistry utilities, including but not limited to PubChem (e.g., pubchem.gov) maintained by the National Institutes of Health (NIH) and the National Cancer Institute, for example, to sort and process the aggregated set of all tentative molecules using underlying the m/z (mass/charge of chemical fragments) ratio data for automatically interpreting close matches across samples. In addition to precisely (but flexibly) grabbing tentative compounds from samples in which they could theoretically exist and preparing the component areas for statistical summary and analysis-including principal component analyses, non-metric multidimensional scaling (NMDS), and/or machine learning algorithms, uafR can also interact with structural data [e.g., in SDF (Structure-Data format)] for all published compounds in the dataset. These data allow detailed summaries of the chemical constituents for each sample to be generated based on the user's chemical(s) of interest. Thus, while a chemical ecologist may be more interested in groups of the relative proportions of alkaloids to polyphenols in a sample, a biochemist may only be interested in steroids groups. These groups (or others) can be selectively pulled from one's dataset to perform follow-up analyses. In addition, researchers (e.g. those performing targeted analysis) that have advanced knowledge of the molecule(s) or functional group(s) of interest can use uafR functions to isolate these chemistries from experimental data, and focus their analysis/interpretation on specified chemicals or chemical groups more generally.

Users may also load personal chemical libraries, as a formatted “.CSV” file using long or wide orientation, to compare any list of chemicals against the set(s) of classifier compounds in their .CSV input library. For the chemical structure processing, the uafR package utilizes Tanimoto similarity, a commonly used and rigorously tested metric for physicochemical comparisons. While there is a broad range of diversity in the chemistry of any system, there exist common structural subunits that can categorize molecules into their potential function(s) and the Tanimoto index provides efficient functional sorting of even diverse chemistries. As an example, these comparisons could be used in agricultural research to rapidly screen plant molecules for insecticidal or repellent properties. More specifically, the Tanimoto similarity metric can be used to discover compounds that will bind known ligands or share biological activity with known drugs. This similarity metric is underapplied, however, because to date, it requires multiple complex steps to generate or acquire data in the appropriate format. The uafR package can harness direct connections between PubChem and R to stream published information on every known (i.e., published and vetted by peer-review for merit) chemical in the dataset. This harnessing of vetted direct connections can bypass the need for other computer programs or coding environments to perform physicochemical comparisons and allows the uafR algorithm to outperform any comparable utility for this stage of mass spectrometry data processing. If a user can install the uafR package and read a “.CSV” file into R, the user will have access to the entirety of PubChem and more.

Data science and informatics can circumvent analytical bottlenecks. Automating the tedious portions of GC-MS data processing can not only turn weeks or months of work into a few keyboard strokes within a day, but can also take human error and subjectivity out of the equation. An efficient and user-friendly tool for interpreting these chemical data is long overdue.

Both the speed and accuracy of GC-MS data processing can be drastically improved with uafR because uafR allows users to fluidly interact with their experiment following tentative library identifications, i.e., after the m/z spectra have been matched against an installed chemical fragmentation database, such as the one maintained by the National Institute of Standards and Technology (NIST), for example. Use of uafR can allow larger datasets to be collected and systematically interpreted quickly. Furthermore, the functions of uafR can allow backlogs of previously collected and annotated data to be processed by new personnel or students as they are being trained. This improved processing is important as we enter the era of exposomics, metabolomics, volatilomes, and landscape level, high-throughput chemotyping. The uafR package was developed to advance collective understanding of chemical data and is applicable to any research that benefits from GC-MS analysis.

uafR was tested in two experimental contexts: (1) A dataset of purified internal standards, which showed that uafR correctly identified the known compounds resulting in R² values ranging from 0.827-0.999 along concentrations ranging from 1×10⁻⁵ to 1×10³ ng/μl, and (2) A large, previously published dataset, where the number and types of compounds identified were comparable (or identical) to those identified with the traditional manual peak annotation process, and NMDS analysis of the compounds produced the same pattern of significance as in the original study.

The following two examples can demonstrate the accuracy and efficiency of uafR. The first example is the identification and analysis of a GC/MS dataset containing samples of a series of four known internal standards at different concentrations. The second example is a re-identification of GC/MS samples from an already published dataset (Ponce M A, Lizarraga S, Bruce A, et al (2022) Grain Inoculated with Different Growth Stages of the Fungus, Aspergillus flavus, Affect the Close-Range Foraging Behavior by a Primary Stored Product Pest, Sitophilus oryzae (Coleoptera: Curculionidae). Environmental Entomology 51:927-939. https://doi.org/10.1093/ee/nvac061). For this dataset, the present inventors compared the same statistical tests for the standardized areas for compounds identified with four methods by the uafR package and those from the published, manual identifications. As described herein, the uafR package can improve chemical workflows in non-GC-MS datasets or meta-analyses.

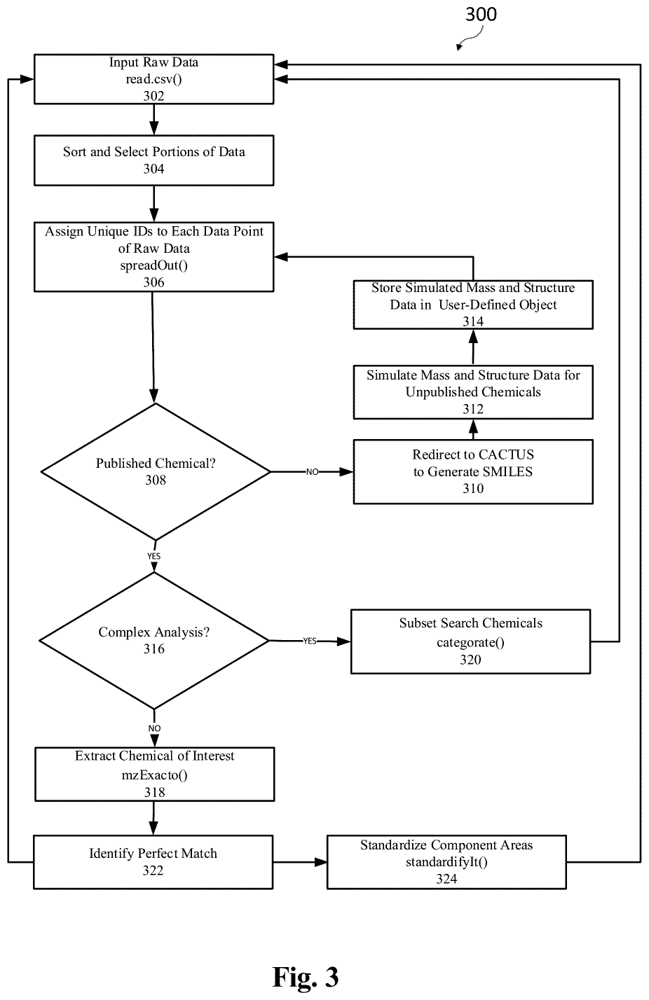

FIG. 3 illustrates an overall exemplary method 300 for identifying the chemical composition of samples, according to an exemplary embodiment of the invention. The method 300 includes several steps. The steps of the method 300 can be completed in any order, depending on particular applications or needs.

uafR is optimized for raw output from Agilent's Unknowns Analysis Software; however, the only aspect of the workflow that is specific to the Unknowns Analysis software are the column names for the input data frame. To briefly describe the output, after setting up the analysis environment (i.e., directing Unknowns Analysis to the sample directory where a “.UAF” file (hence, uafR) is created), running a deconvolution algorithm to identify peaks, and searching the peaks against an installed library (blank subtraction and target matching are also options and will not affect the input for uafR), a single “.CSV” file containing basic GC-MS output (i.e., retention times, peak area, captured mass-to-charge ratios (m/z), compound name, match quality) and a sample origin identifier (i.e., sample name or file name) for tentative compounds across all samples can be exported and read into R using a uafR function entitled “read.csv( )” (step 302 in FIG. 3). After reading the data into R and loading the package, uafR can use published information to sort and precisely select portions of the data that the user may be interested in (step 304).

The first function for GC-MS data is entitled “spreadOut( )” (step 306). Running the spreadOut( ) function on the properly formatted GC-MS input can automatically prepare the data for the next steps in the processing pipeline. Briefly, the spreadOut( ) function takes every recorded data point for every sample and expands the recorded data points into large database formats with unique identifiers assigned for each data point. The unique identifiers (unique IDs) are automatically created from the input data and are used to extract specific values from the raw recorded data. In addition to setting up large databases containing component area, tentative compound identities, match factors, captured m/z values, retention time indices, sample identities, and the unique IDs, the spreadOut( ) function can also communicate with online databases to download relevant information about every tentative compound. For published chemicals, this relevant information can include exact mass, m/z histograms, and every name the chemical has, etc. (step 308, path “Yes”).

Instances in which the chemical cannot be identified by name on PubChem (step 308, path “No”) can be redirected (step 310) to CADD Group Chemoinformatics Tools and User Services (CACTUS, https://cactus.nci.nih.gov/) from which a canonical Simplified Molecular Input Line Entry System (SMILES) can be generated using that server and algorithm The SMILES notation is then used to simulate the mass and structure data for, as-of-yet unpublished chemicals on PubChem (step 312). All this information, including the large databases, are stored in the local memory (e.g., in the user's computer) as a user-defined object list (step 314). Subsequent functions are designed to seamlessly interact with the list and will automatically use relevant information collected during execution of spreadOut( ) (step 306).

The next step in the GC-MS workflow will depend on the type of analysis the user is performing. If the chemicals of interest are already known and no complex datasets or analyses are needed (step 316, path “No”), the chemicals of interest can be extracted by name with a single function entitled “mzExacto( )” (step 318). However, for complex datasets or analyses that involve more unknowns (step 316, path “Yes”), the user may want to cast a broader, but still accurate, net. There are multiple steps that can be taken to focus on the most relevant chemicals in a dataset using the features of uafR.

A simple and effective approach is to subset the search chemicals by setting a minimal match factor on the raw output of Unknowns Analysis (or other GC-MS software) (step 320). This can be done with R code described in the vignette published with the package (Appendix A; Appendix B: Vignettes—uafR.rmd).

Another approach can include subsetting with output from the function entitled “categorate( )” This function also uses PubChem to communicate with online databases and generate categorically, structurally, and chemically identifying information for every published chemical in the dataset. The categorical data include whether the chemical is biologically derived [Natural Products Online database (LOTUS; https://lotus.naturalproducts.net/)], has flavor or smell [Flavor and Extract Manufacturers Association (FEMA; https://www.femaflavor.org/)], has varied biological activities [Kyoto Encyclopedia of Genes and Genomes (KEGG; https://www.genome.jp/kegg/)], medical subject headings (MeSH; https://www.nlm.nih.gov/mesh/), or other information about their reactivity [Food and Drug Administration-Structured Product Labeling (FDA/SPL; https://www.fda.gov/) and Reactive Groups from PubChem (https://pubchem.ncbi.nlm.nih.gov/)].

After the categorical information is collected, the function generates substructure data for the chemicals to also be subsetted by common functional groups. The generated substructure information includes the number of rings, all subgroups (e.g., R—COH, R—COOH, etc.) and their counts, all atoms (e.g., C, N, S, As, etc.) and their counts, and the number of charges for every chemical with published structural data (or canonical SMILES from CACTUS) on PubChem. The final steps in categorate( ) will not only assist in subsetting compounds of interest for extracting from GC-MS datasets, but can also be used to perform meta-analyses on published chemistries.

In order to run categorate( ) users are required to include an input library that contains columns with labeled chemicals. The chemical labels are customizable, but a preferred approach is to label a set of chemicals by a common feature or biological activity. For example, if a researcher has a set of plant chemicals of interest to test against active ingredients in pharmaceuticals, the input library could contain n columns whose headings are the biological activity (e.g., diuretic, blood pressure, etc.) and the contents (rows under the heading) are the active chemicals used in products that are approved for those medical outcomes. The categorate( ) function will then take the input library (saved as a “.CSV”) and compare every chemical of interest to the chemicals in each user-defined “chemical category,” returning two additional data frames—(1) whether it has a strong (Tanimoto similarity greater than 0.95) or moderate (greater than 0.85) structural match with any of the chemicals in each group; and (2) for strong matches, the name of a chemical that it was most similar to.

The utility of this information and approach cannot be overstated. For chemistry, structure defines function, so identifying structural matches is effectively identifying chemicals with the same function. This not only provides a powerful tool for novel chemical activity discoveries and/or natural backups to synthesized chemistries, but can also allow researchers to subset GC-MS data by general chemical structures or activities they are interested in. The possibilities are limited only by the maximum file size a user can create in the specified “.CSV” format and whether structural data were able to be generated from PubChem for the chemical(s). Subsetting of information generated with categorate( ) can be done using the function entitled “exactoThese( )” Users can specify which set of information they would like to subset and indicate desired criteria the chemicals should meet.

Next in the GC-MS workflow is to put the published information to use and aggregate every occurrence of the user-specified chemicals across every GC-MS sample. “mzExacto( )” takes the output from spreadOut( ) along with the list of chemical names, and returns a single data frame containing their optimal retention time, exact mass, best identified match factor, and aggregated component area across samples in which it occurs (0 when absent) (step 322). Additional technical details for this algorithm are available with the package (github.com/castratton/uafR). Briefly, after collecting mass and m/z information for the input chemicals of interest, they are ordered by exact masses so likely retention time windows can be determined based on the general structure of the input data and the information stored from “spreadOut( )” After identifying perfect matches (i.e., those with high match factors and the same chemical names) the algorithm looks again through each sample for instances where the top two published m/z values for the tentative identity are the same as the query chemicals of interest. These matches are based on standard manual approaches to resolve uncertainties in any complex GC/MS workflow. The m/z values within retention time windows generated by the input data must be similar enough that the chemical fragments are practically and theoretically identical.

At this point in the GC/MS workflow, the most common step is to standardize component areas for tentatively identified chemicals by quantifying their values relative to known internal or external standard(s) (step 324). The function entitled “standardifyIt( )” takes the output from mzExacto( ) and either a user-specified internal standard (e.g., tetradecane, or user defined-internal standard) or calibration curves (raw values) from an external standard(s), along with sub-arguments that allow the standardization to be tuned to the experimental methods. standardifyIt( ) returns a data frame that is standardized relative to the known chemical distributions and formatted for subsequent statistical analyses. Common statistical protocols for GC-MS data include ordination analyses (e.g., PCA, NMDS, etc.), multivariate statistical tests (e.g., ANOSIM, MANOVA, PERMANOVA, etc.) and/or deep learning (neural networks or machine learning). Each of the required formats for running these statistics on GC-MS data are easily achievable with the final output of mzExacto( ) and standardifyIt( ).

Beyond automating a process that can require hours of work per sample, with potentially hundreds of samples per study, the uafR package described herein makes cheminformatics a possibility for anyone working with GC-MS or chemical identity data. Furthermore, the public databases uafR accesses will only improve in data quality/quantity with time and increased use. To showcase the utility and validity of uafR for GC-MS workflows, two datasets were analyzed-one containing a set of known standards pipetted in known quantities across three samples (low, medium, and high concentrations) and the other, consisting of a recently published set of 35 samples.

As another example, the uafR software can be embedded within a system designed to facilitate cheminformatics. Such an example system includes a fluid chromatographer. The fluid chromatographer may be any chromatographer, such as an Agilent gas chromatograph, for example. The fluid chromatographer includes a mass spectrometer, which may be any mass spectrometer, such as an Agilent mass spectrometer, for example. The fluid chromatographer, or the constituent mass spectrometer is configured to accept a sample and produce a sample chromatogram. The sample chromatogram describes at least a portion of the sample, the portion comprised of one or more compounds to be identified. The sample chromatogram includes a plurality of sample mass spectrograms.

The fluid chromatographer and mass spectrometer may be completely functionally overlapping, meaning the fluid chromatographer may only be capable of performing the tasks of the mass spectrometer, and a sample chromatogram produced by such a fluid chromatographer would include a single mass spectrogram. Alternatively, such a fluid chromatographer could be run multiple time on the same sample or portion of the sample, thereby approximating a plurality of mass spectrograms as a chromatogram as would be produced by a conventional fluid chromatographer.

The system also includes a processor, an output coupled to the processor, and a memory coupled to the processor. The processor and memory may be divided across one or more discrete processors and memory devices: for example, a processor and memory may be embedded within or directly coupled to the fluid chromatographer, while another processor and memory may be embedded within a networked computing device, physically separated from the fluid chromatographer.

The processor serves to perform various operations, for example, in accordance with instructions or programming executable by the processor. Although the processor may be configured by use of hardwired logic, typical processors are general processing circuits configured by execution of programming. The processor includes elements structured and arranged to perform one or more processing functions, typically various data processing functions. Although discrete logic components could be used, the examples utilize components forming a programmable CPU. The processor for example includes one or more integrated circuit (IC) chips incorporating the electronic elements to perform the functions of the CPU. The processor for example, may be based on any known or available microprocessor architecture, such as a Reduced Instruction Set Computing (RISC) using an ARM architecture, as commonly used today in mobile devices and other portable electronic devices. Of course, other processor circuitry may be used to form the CPU or processor hardware. Although the illustrated examples of the processor include only one microprocessor, for convenience, a multi-processor architecture can also be used. A digital signal processor (DSP) or field-programmable gate array (FPGA) could be suitable replacements for the processor but may consume more power with added complexity.

A memory is coupled to the processor. The memory is for storing data and programming. In the example, the memory may include a flash memory (non-volatile or persistent storage) and/or a random-access memory (RAM) (volatile storage). The RAM serves as short term storage for instructions and data being handled by the processor e.g., as a working data processing memory. The flash memory typically provides longer term storage.

Of course, other storage devices or configurations may be added to or substituted for those in the example. Such other storage devices may be implemented using any type of storage medium having computer or processor readable instructions or programming stored therein and may include, for example, any or all of the tangible memory of the computers, processors or the like, or associated modules.

The system may also include a network interface coupled to the processor. The system may be implemented in a distributed manner: the processor may be divided in to two or more processors, along with two or more memory devices. The processors may work in parallel, and may also specialize and perform particular tasks. The memory devices may store a full copy of all data, or may specialize and store particular data relevant to a particular processor. In an example, the system is divided into a local and remote grouping. A local processor, local memory, and local network interface can be directly coupled to the fluid chromatographer; while a remote processor, remote memory, and remote network interface can receive the plurality of mass spectrogram data and perform data warehousing. As used herein, the term “system” may refer to distributed and non-distributed systems and may interchangeably be referred to as a computer system or computing device. The corresponding functions relating to the devices and systems as described herein may also be articulated as computer-implemented methods to be performed without limitation to any particular type of computer system or computing devices and/or in the form of computer instructions stored in a non-transitory machine readable medium programmed with instructions stored in the non-transitory machine-readable medium.

Receiving or transmitting data can include digital network communication, electronic signaling, or physical analog communication e.g., fingers typing on a keyboard and eyes receiving information from a digital display.

The memory can also include an idealized mass spectrogram library, such as the library used in Agilent's Unknowns analysis peak detection software, for example. The idealized mass spectrogram library include a plurality of idealized mass spectrograms, and each idealized mass spectrogram of the plurality of idealized mass spectrograms associated with an idealized compound.

The memory can further include a reference compound library, such as the NIST chemical fragmentation database, for example. The reference compound library can include a plurality of reference compound identifiers, each reference compound identifier of the plurality of reference compound identifiers being associated with a reference structural datum.

The memory further includes programming that can include the uafR package, the R software to run the uafR package, and any additional programming needed to run the fluid chromatographer, for example. The programming may be distributed as required by different devices in the system (i.e., the fluid chromatographer). Though the uafR package is described as using R, any software language, compilation, instruction set, or programming capable of describing the functionality of the programming is contemplated.

In this example, execution of the programming by the processor configures the system to perform functions. The system matches a sample mass spectrogram of the plurality of sample mass spectrograms to one or more tentative idealized mass spectrograms of the plurality of idealized mass spectrograms. This matching may be performed within the fluid chromatography device, or by software, such as the Agilent Unknowns analysis software, for example. The system also matches the idealized compound identifier associated with a tentative idealized mass spectrogram of the one or more tentative idealized mass spectrograms to a matching reference compound identifier of the plurality of reference compound identifiers. This matching may be performed with a function pipeline including spreadOut( ) and mzExacto( ) The system also accepts a filtering structural datum, which would become part of the input into the exactoThese( ) function. The system further selects the matching reference compound identifiers matched to the idealized compounds associated with the one or more tentative idealized mass spectrograms with the reference structural datum satisfying the filtering structural datum, utilizing the categorate( ) and exactoThese( ) functions. The system still further outputs, via the output, the selected matching reference compound identifiers corresponding to the one or more compounds to be identified.

An output or interface for performing the functions as described may be, for example and without limitation, a touchscreen device where instructions are inputted via a user interface application through manipulation or gestures on a touch screen. For output purposes, the touch screen of the user interface and file intake includes a display screen, such as a liquid crystal display (LCD) or light emitting diode (LED) screen or the like. For input purposes, a touch screen includes a plurality of touch sensors.

In other embodiments, a keypad may be implemented in hardware as a physical keyboard of the user interface and file intake, and keys may correspond to hardware keys of such a keyboard. Alternatively, some or all of the keys (and keyboard) may be implemented as “soft keys” of a virtual keyboard graphically represented in an appropriate arrangement via touch screen. The soft keys presented on the touch screen may allow the user to invoke the same functions as with the physical hardware keys. The output or interface is not limited to any particular hardware and/or software for facilitating user input. The user interface and file intake may have a graphical interface, such as a screen, and tactile interfaces, like a keyboard or mouse. It may also have a command line interface that allows for text input commands. The interface and may also have a port to accept a connection from an electronic device. The output may include a printer to produce a printed readout of the outputted information.

The system can also accept a personal compound library, the personal compound library (e.g., the personal chemical libraries, .CSV input library) including a plurality of personal compounds, each personal compound of the plurality of personal compounds associated with a personal structural datum, using a function entitled “personalLib( )” for example. The system can further match the idealized compound associated with a tentative idealized mass spectrogram of the one or more tentative idealized mass spectrograms to a matching personal compound of the plurality of personal compounds, using spreadOut( ) and mzExacto( ) The system can still further select the matching personal compounds matched to the idealized compounds associated with the one or more tentative idealized mass spectrograms with the personal structural datum satisfying the filtering structural datum, using categorate( ) and exactoThese( ) The system can yet further output, via the output, the selected matching personal compounds.

The first dataset on which uafR functions were tested was a series of samples of standards including, ethyl hexanoate (Prod #14896, CAS #123-66-0, Millipore Sigma, Burlington, MA, USA), methyl salicylate (Prod #M6752, CAS #119-36-8, Millipore Sigma), octanal (Prod #S7303001712, CAS #124-13-0, Merck, Darmstadt, Germany), and undecane (Prod #S7466429734, CAS #1120-21-4, Merck, Darmstadt, Germany). These samples of standards were prepared in a serial dilution using 1 mL of neat compound from each standard and diluting in 10 mL in dichloromethane, and subsequently moving 1 mL of the dilutant to a new container with 10 mL of fresh dichloromethane until the following amounts of ethyl hexanoate, methyl salicylate, octanal, and undecane were achieved: low (0.000087 ng, 0.0001179 ng, 0.000082 ng, and 0.000072 ng, respectively), medium (434.5 ng, 589.5 ng, 410 ng, and 370 ng, respectively), or high (2172.5, 2947.5 ng, 2050 ng, and 1850 ng, respectively) relative quantities.

The second dataset was GC/MS data collected on an Agilent 7890b gas chromatograph (GC) equipped with an Agilent Durabond HP-5 column (30 m length, 0.250 mm diameter, and 0.25 μm film thickness) using He as the carrier gas at a constant 5 ml/min flow and 39 cm/s velocity, coupled with an Agilent 5997B mass spectrometer (MS) single quadrupole detector that had been manually processed and published. Briefly, the samples were collected from grain samples that were a) UV sterilized (negative control), b) clean grain from storage (positive control), c) inoculated with asexual fungal spores, d) inoculated with sexual fungal spores (see Ponce et al. 2022).

After analyzing the samples on the GC/MS, the raw output was saved to a local directory and loaded into Unknowns Analysis following default protocols. After loading the samples and loading the methods file to every sample, the Unknowns Analysis deconvolution algorithm identified the most accurate peaks for every chromatogram. Each peak was then searched against the NIST 20 database. The aggregated data frame was exported as a “.CSV” file. This data frame included columns for the compound names (“Compound. Name”), the file name of the file the tentative identity is from (“File.Name”), top m/z peaks captured by the GC/MS (“Base.Peak.MZ”), match factors for tentative identities (“Match.Factor”), and retention times (“Component.RT”).

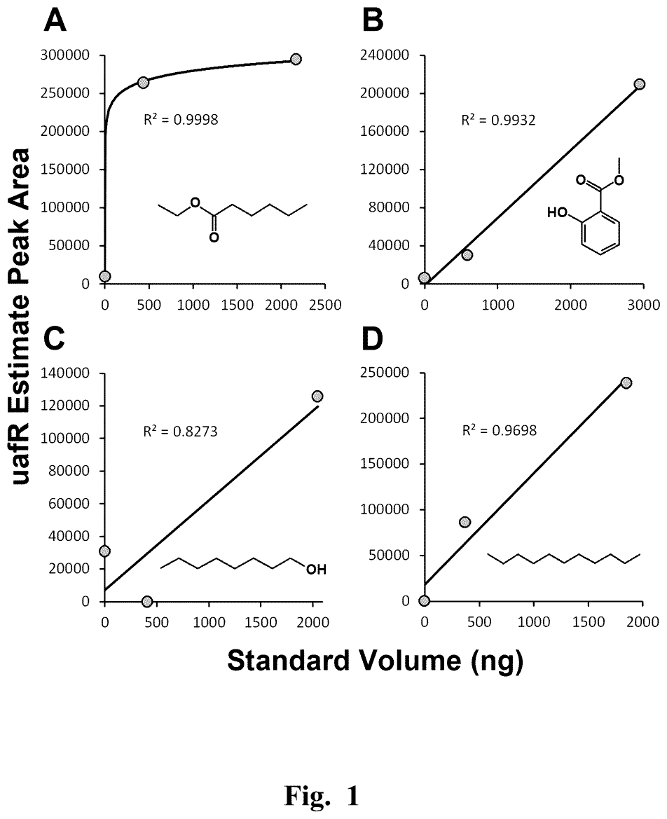

FIG. 1 illustrates correlations between volume of standards tests via GC/MS and the peak area estimates generated by uafR for: (A) Ethyl hexanoate, (B) Methyl salicylate, (C) Octanal, and (D) Undecane. Points represent raw data while the line represents a natural log fit (A) or linear fit (B or D) to the raw data.

Peak areas calculated by uafR for the set of standards correlated with the volume of the standards injected, with R² values ranging from 0.8273 to 0.9998 (FIG. 1). Importantly, the single standard (e.g., octanal) with a lower correlation coefficient was likely misread by the MS or had volatized prior to being run on the GC-MS. It is known that octanal volatilizes very easily, and is used by plants as an anti-fungal compound to protect fruit.

After confirming that uafR can precisely identify chemicals that are known to be in a sample, the next step was to assess its accuracy in a more complex experiment with unknowns. Using raw GC-MS data from a recently published experiment allowed the workflow to be tested against a peer-reviewed study. uafR was able to identify the manually selected compounds with precise matches to manually identified retention times (Table 1) and yielded the same overall pattern of significance in ANOSIM analysis (Table 2). The true benefit of using uafR is not merely found in the accuracy of uafR, but rather found in the fact that analyzing this entire experiment took 150 minutes of automated computation using a standard desktop computer with a 3.30 GHz processor and 16 GB RAM. For context, the original manual identifications required months of labor.

TABLE 1
Summary of NMDS and ANOSIM calculation
for models processed with uafR.

| | NMDS | ANOSIM | | # Compounds |
|---|---|---|---|---|
| Model | Stress | R | P | in Final Table |
| Original Ponce et al. | 0.10 | 0.20 | 0.001 | 33 |
| 2022 | | | | |
| uafR Ponce et al. | 0.11 | 0.185 | 0.009 | 33^{a} |
| 2022 | | | | |
| >65% Match Factor | 0.17 | 0.068 | 0.17 | 427 |
| >75% Match Factor | 0.11 | 0.016 | 0.33 | 116 |
| >88.9 Match Factor | 0.15 | 0.034 | 0.29 | 30 |
| >97.2 Match Factor | 0.04 | −0.034 | 0.58 | 3 |

^{a}Only 30 compounds used for analysis, since three compounds were not present in enough experimental replicates

TABLE 2
Chemicals identified in Ponce et al. 2022 using manual identification,
versus compounds identified by the uafR package using the same selection
criteria: >75% match of the chemical ID, and present in more than one sample.
Compounds shared between identification techniques are in bold print.

| Ponce et al. 2022 | | uafR | |
|---|---|---|---|
| Chemical ID | RT | Chemical ID | RT |
| Pivalaldehyde, semicarbazone | 4.735 | | |
| 2-Butenal | 4.788 | | |
| 2,4-Dimethyl-1-heptene | 4.792 | 2,4-Dimethyl-1-heptene | 4.796 |
| 2-Pentanone, 4-hydroxy-4- | 4.802 | 2-Pentanone, 4-hydroxy-4- | 4.804 |
| methyl | | methyl- | |
| | | Cyclopentanone, 2-methyl- | 5.513 |
| Benzene, propyl | 6.292 | | |
| | | Benzene, 1-ethyl-3-methyl- | 6.404 |
| 1-Octen-3-ol | 6.516 | | |
| Butanal | 6.525 | | |
| 4,5-Dichloro-1,3-dioxolan-2- | 6.628 | Benzene, 1-ethyl-4-methyl- | 6.633 |
| one | | | |
| 3-Octanone | 6.648 | 3-Octanone | 6.645 |
| Decane | 6.823 | | |
| Mesitylene | 6.869 | Mesitylene | 6.872 |
| Benzene, 1,2,4-trimethyl- | 7.319 | Benzene, 1,2,4-trimethyl- | 7.017 |
| D-Limonene | 7.364 | | |
| Benzeneethanol, beta-ethyl | 7.541 | | |
| Benzene, 1,4-diethyl | 7.659 | Benzene, 1,2-diethyl- | 7.418 |
| Benzene, 1,2-diethyl | 7.759 | Benzene, 1,4-diethyl- | 7.738 |
| 1,3,8-p-Menthatriene | 7.86 | Limonene | 7.780 |
| | | p-Cymene | 8.200 |
| | | Benzene, 2-ethyl-1,4-dimethyl- | 8.203 |
| 4-Dichloromethyl-2[[2-[1- | 8.271 | | |
| methyl-2- | | | |
| pyrrolidinyl]ethyl]amino-6- | | | |
| Trichloromethylpyrimidine | | | |
| Benzene, (2-methyl-1- | 8.279 | | |
| propenyl)- | | | |
| 1-Phenyl-1-butene | 8.282 | | |
| Linalool | 8.314 | Linalool | 8.315 |
| | | Undecane | 8.329 |
| Nonanal | 8.37 | Nonanal | 8.373 |
| 2-Thiophenecarboxylic acid, 5- | 9.044 | Cyclopentasiloxane, | 8.950 |
| nonyl- | | decamethyl- | |
| Dichloroacetaldehyde | 9.735 | | |
| | | Cyclopentanecarboxylic acid, | 10.185 |
| | | pentyl ester | |
| Linalyl acetate | 10.558 | 1,6-Octadien-3-ol, 3,7- | 10.559 |
| | | dimethyl-, formate | |
| Beta-Ocimene | 10.561 | | |
| 2-Thiophenecarboxylic acid | 10.59 | 2-Thiophenecarboxylic acid, 3- | 10.590 |
| | | methylbutyl ester | |
| 1-Pent-3-ynylcyclopenta-1,3- | 10.653 | | |
| diene | | | |
| | | Ethanone, 1-(2,5- | 10.816 |
| | | dimethylphenyl)- | |
| 1,5,6,7- | 10.822 | Ethanone, 1-(3,4- | 10.821 |
| Tetramethylbicyclo[3.2.0]hepta- | | dimethylphenyl)- | |
| 2,6-diene | | | |
| | | Ethanone, 1-(2,4- | 10.916 |
| | | dimethylphenyl)- | |
| Ethanone, 1-(4-ethylphenyl) | 11.108 | Ethanone, 1-(4-ethylphenyl)- | 10.973 |
| | | Cyclotetrasiloxane, | 13.749 |
| | | octamethyl- | |
| Butyl citrate | 21.812 | | |
| 1-Methyl-4-phenyl-5-Thioxo- | 23.957 | | |
| 1,2,4-triazolidin-3-one | | | |
| 9-Octadecenamide, (Z-) | 26.311 | | |

As another example, the uafR software can be embedded within systems designed to use Gas Chromatography-Mass Spectrometry. Such systems can include instruments with Electron Ionization (EI) or Chemical Ionization (CI), for example. Electron Ionization produces positively charged ions by knocking off electrons from sample molecules. These ions go to the mass analyzer where m z ratios are determined. Chemical Ionization uses an ionized reagent gas to indirectly ionize the sample. It produces fewer fragments that are also sent to mass analyzer for m z ratios to be determined.

Other exemplary systems designed to use Gas Chromatography-Mass Spectrometry can include Quadrupole, Time of Flight, Ion Trap, Magnetic Sector, and Hybrid mass analyzers, for example. Quadrupole sorts ions by specific m z ratios so they can be directed to the mass analyzer sequentially. Time-of-flight accelerates ions into a flight tube where they are separated by velocity to the mass analyzer. Ion trap stops ions in an electrostatic field and ejects them based on m z ratio so they can be sent to the mass analyzer sequentially. Magnetic sector deflects ions using a magnetic field. The curve of the ion's travel allows them to be separated by m z ratios. Hybrid mass analyzers include combinations of the different mass analyzers to further improve the separation and detection of m z ratios.

As another example, the uafR software can be embedded within systems designed to use Liquid Chromatography-Mass Spectrometry. Such systems can include instruments with Electrospray Ionization (ESI) and Atmospheric Pressure Chemical Ionization (APCI), for example. Electrospray Ionization involves dissolving the sample in a solvent and spraying it through a needle with high voltage. It makes a fine mist of charged droplets. Converted to gas via evaporation and rapid introduction of positive charge. The ions travel to the mass analyzer under atmospheric pressure. Atmospheric Pressure Chemical Ionization uses a high voltage to create a strong electric field. The sample travels to the field and becomes ionized without breaking into a lot of fragments. These ions are guided to the mass analyzer under atmospheric pressure.

Other exemplary systems designed to use Liquid Chromatography-Mass Spectrometry can include Quadrupole, Time of Flight, Ion Trap, Orbitrap, Magnetic Sector, and Hybrid mass analyzers, for example. Orbitrap uses an electrostatic field between two electrodes to trap ions and measure their oscillation frequencies. These frequencies provide high-resolution m z measurements.

As another example, the uafR software can be embedded within systems designed to use Tandem Mass Spectrometry. Such systems can include, for example, two mass analyzers-ions read/selected by first analyzer, then fragmented in an additional collision cell, and m z recorded by second mass analyzer.

As another example, the uafR software can be embedded within systems designed to use High-Resolution Mass Spectrometry. Such systems can use Time-of-Flight, Orbitrap, of Fourier Transform Ion Cyclotron Resonance (FT-ICR), for example. Fourier Transform Ion Cyclotron Resonance (FT-ICR) uses a magnetic field to force ions into circular orbits. The motion depends on the m z ratio. As ions return to original motion they emit signals. These signals are recorded and are directly related to the m z ratios.

The uafR uses the recorded m z ratios for published molecules to pick out component areas for chemicals across all samples in an experiment. Since the result for each of the referenced technologies is the m z spectra for every molecule, uafR can apply to the output. The only limiting factor is the availability of published spectral data on PubChem for the different instruments/techniques. As PubChem's server is populated with more spectral data, uafR can be adapted to use the updated results.

The possible applications of a direct connection between R and PubChem are limitless. Beyond statistical tests and advanced computational pipelines, the graphical framework can provide publication quality visuals with minimal code. This package harnesses the most advanced open-source chemical dataset and makes it accessible to anyone with basic experience working in R.

The described workflow and package utilities of uafR bring GC-MS data processing up to par with the advanced technology that generates the data. Anyone with the ability to install the uafR package and load a “.CSV” file into R now has access to a suite of functions that streamline a complex workflow so more effort can be spent interpreting rather than preparing data.

Chemical knowledge is more advanced and accessible than at any point in history. The precision of GC-MS instruments and, consequently, their output, allows published information to be accessed with 100% accuracy. While previous algorithms have focused on using statistics to separate likely aggregates of compound areas, their accuracy fails in complex contexts because too many distinctly different chemicals “behave” (i.e., have the same mass and/or retention indices) the same and therefore cannot be teased out statistically without additional knowledge.

uafR is the first and, to date, only R package that uses published data to extract the most accurate compound areas for the most likely compound identifications. By automating this component of the GC-MS workflow, uafR can greatly increase the speed at which chemistry datasets are published, the size of chemical studies that can be conducted, and the accessibility of chemical analyses to scientists in related fields.

Although the invention is illustrated and described herein with reference to specific embodiments, the invention is not intended to be limited to the details shown. Rather, various modifications may be made in the details within the scope and range of equivalents of the claims and without departing from the invention.

What is claimed is:

1. A system comprising:
a fluid chromatographer including a mass spectrometer, configured to accept a sample and produce a sample chromatogram describing at least a portion of the sample comprised of one or more compounds to be identified, the sample chromatogram including a plurality of sample mass spectrograms;
a processor;
an output, coupled to the processor;
a memory, coupled to the processor, the memory including:
an idealized mass spectrogram library, the idealized mass spectrogram library including a plurality of idealized mass spectrograms, each idealized mass spectrogram of the plurality of idealized mass spectrograms associated with an idealized compound;
a reference compound library, the reference compound library including a plurality of reference compound identifiers, each reference compound identifier of the plurality of reference compound identifiers associated with a reference structural datum; and
programming in the memory;
wherein execution of the programming by the processor configures the system to perform functions, including functions to:
match a sample mass spectrogram of the plurality of sample mass spectrograms to one or more tentative idealized mass spectrograms of the plurality of idealized mass spectrograms;
match the idealized compound identifier associated with a tentative idealized mass spectrogram of the one or more tentative idealized mass spectrograms to a matching reference compound identifier of the plurality of reference compound identifiers;
accept a filtering structural datum;
select the matching reference compound identifiers matched to the idealized compounds associated with the one or more tentative idealized mass spectrograms with the reference structural datum satisfying the filtering structural datum; and
output, via the output, the selected matching reference compound identifiers corresponding to the one or more compounds to be identified.

2. The system of claim 1, wherein execution of the programming by the processor further configures the system to perform functions, including functions to:
match each sample mass spectrogram of the plurality of sample mass spectrograms to one or more tentative idealized mass spectrograms of the plurality of idealized mass spectrograms.

3. The system of claim 1, wherein execution of the programming by the processor further configures the system to perform functions, including functions to:
match the idealized compound associated with each tentative idealized mass spectrogram of the one or more tentative idealized mass spectrograms to a matching reference compound of the plurality of reference compounds.

4. The system of claim 1, wherein execution of the programming by the processor further configures the system to perform functions, including functions to:
accept a personal compound library, the personal compound library including a plurality of personal compounds, each personal compound of the plurality of personal compounds associated with a personal structural datum;
match the idealized compound associated with a tentative idealized mass spectrogram of the one or more tentative idealized mass spectrograms to a matching personal compound of the plurality of personal compounds;
select the matching personal compounds matched to the idealized compounds associated with the one or more tentative idealized mass spectrograms with the personal structural datum satisfying the filtering structural datum;
output, via the output, the selected matching personal compounds.

5. The system of claim 4, wherein execution of the programming by the processor further configures the system to perform functions, including functions to:
process the personal compound library using Tanimoto similarity.

6. The system of claim 1, wherein execution of the programming by the processor further configures the system to perform functions, including functions to:
match a sample mass spectrogram of the plurality of sample mass spectrograms to one or more tentative idealized mass spectrograms of the plurality of idealized mass spectrograms based on underlying mass/charge of chemical fragments ratio data.

7. The system of claim 1, wherein the function for matching the idealized compound identifier associated with a tentative idealized mass spectrogram of the one or more tentative idealized mass spectrograms to a matching reference compound identifier of the plurality of reference compound identifiers further comprises:
the idealized compound identifier configured to include an idealized compound name;
the reference compound identifier configured to include a reference compound name;
matching the idealized compound identifier to the reference compound identifier utilizes a match between the idealized compound name and the reference compound name when the idealized compound name is included within the idealized compound identifier;
the idealized compound identifier further configured to include an idealized Simplified Molecular Input Line Entry System (SMILES) object;
the reference compound identifier further configured to include a reference SMILES object; and
matching the idealized compound identifier to the reference compound identifier utilizes a match between the idealized SMILES object and the reference SMILES object when the idealized compound name is a null value within the idealized compound identifier.

8. The system of claim 1, wherein the function for matching the idealized compound identifier associated with a tentative idealized mass spectrogram of the one or more tentative idealized mass spectrograms to a matching reference compound identifier of the plurality of reference compound identifiers further comprises:
generating an idealized substructure dataset associated with the idealized compound identifier based upon one or more reference substructure datasets associated with the matching reference compound identifier.

9. The system of claim 1, wherein execution of the programming by the processor further configures the system to perform functions, including functions to:
standardize the reference structural datum satisfying the filtering structural datum; and
output, via the output, the selected matching reference compound identifiers corresponding to the one or more compounds to be identified and the standardized reference structural datum satisfying the filtering structural datum.

10. A system comprising:
a processor;
a network interface;
an output, coupled to the processor;
a memory, coupled to the processor, the memory including:
an idealized mass spectrogram library, the idealized mass spectrogram library including a plurality of idealized mass spectrograms, each idealized mass spectrogram of the plurality of idealized mass spectrograms associated with an idealized compound;
a reference compound library, the reference compound library including a plurality of reference compounds, each reference compound of the plurality of reference compounds associated with a reference structural datum; and
programming in the memory;
wherein execution of the programming by the processor configures the system to perform functions, including functions to:
receive a sample chromatogram describing a sample, the sample chromatogram including a plurality of sample mass spectrograms;
send, via the network interface, a sample mass spectrogram of the plurality of sample mass spectrograms;
receive, via the network interface, one or more tentative idealized mass spectrograms, the one or more tentative idealized mass spectrograms matching a sample mass spectrogram of the plurality of sample mass spectrograms;
send, via the network interface, a tentative idealized mass spectrogram of the one or more tentative idealized mass spectrograms;
receive, via the network interface, an idealized compound, the idealized compound matching the tentative idealized mass spectrogram;
accept a filtering structural datum;
send, via the network interface, the idealized compound and the filtering structural datum;
receive, via the network interface, a selected matching reference compound, the selected matching reference compound matched to the idealized compound and satisfying the filtering structural datum; and
output, via the output, the selected matching reference compounds.

11. A method for identifying one or more compounds, the method comprising:
matching a sample mass spectrogram of a plurality of sample mass spectrograms to one or more tentative idealized mass spectrograms of a plurality of idealized mass spectrograms;
matching an idealized compound identifier associated with a tentative idealized mass spectrogram of the one or more tentative idealized mass spectrograms to a matching reference compound identifier of a plurality of reference compound identifiers;
accepting a filtering structural datum;
selecting the matching reference compound identifiers matched to the idealized compounds associated with the one or more tentative idealized mass spectrograms with the reference structural datum satisfying the filtering structural datum; and
outputting, via an output, the selected matching reference compound identifiers corresponding to the one or more compounds to be identified.
